(19) United States
(12) Patent Application Publication
(10) Pub. No.: US 2025/0122243 A1
(43) Pub. Date: Apr. 17, 2025

(54) PROTEIN BASED VACCINE AND PRODUCTION METHOD THEREOF FOR SARS COV-2

(71) Applicant: Prime Bio, Inc., North Dartmouth, MA (US)

(72) Inventors: Bal Ram Singh, Dartmouth, MA (US); Raj Kumar, Dartmouth, MA (US)

(73) Assignee: Prime Bio, Inc., North Dartmouth, MA (US)

(21) Appl. No.: 18/485,300

(22) Filed: Oct. 11, 2023

(51) Int. Cl. C07K 14/005; C12N 5/071; C12N 15/65; C12N 15/85

(52) U.S. Cl. CPC C07K 14/005; C12N 5/0682; C12N 15/65; C12N 15/85

(57) ABSTRACT

The present invention is directed to a composition, mammalian cell and vector, and a method for the production of, and method of treatment with, a recombinant protein vaccine in a mammalian cell line. The methods and compositions are particularly useful for generating the stable expression of a recombinant protein vaccine of interest. The invention is particularly useful for the production of vaccines to aid in protection against viral pathogens for vertebrates, in particular mammalians, especially humans. The mammalian cell for producing a protein of interest comprises: a plasmid encoded with a nucleotide sequence encoding one or more epitopes or subunits of the SARS-CoV-2 that are embedded in the nucleotide sequence encoding a detoxified recombinant tetanus toxin (DrTeNT).

### REFERENCE TO SEQUENCE LISTING SUBMITTED ELECTRONICALLY

The official copy of the sequence listing is submitted electronically via the Patent Center as an XML file in compliance with the ST26 standard named “T2 Process_sequence ID1 listing.xml” created on Sep. 13, 2023, and having a size of 3 kilobytes. The sequence listing is filed concurrently with the specification. The sequence listing is part of the specification and is incorporated by reference herein in its entirety.

### FIELD OF THE INVENTION

Present invention is directed to a composition and a method for the production of a recombinant protein vaccine in a mammalian cell line, and methods of treatment The methods and compositions are particularly useful for generating the stable expression of a recombinant protein vaccine of interest, such as a vaccine against COVID-19. The invention is particularly useful for the production of vaccines to aid in protection against viral pathogens for vertebrates, in particular mammalians, especially humans.

### BACKGROUND OF THE INVENTION

The SARS-CoV-2 virus is an RNA virus that belongs to the Coronaviridae family and causes COVID-19 infection. Currently there is no effective treatment available for Covid infection. The medical management is totally dependent on indications and the severity of the infection. There are several vaccines available to prevent the spread and the severity of the infection to some extent. These vaccines received emergency use authorizations from various drug approval authorities, including FDA, and are mainly based on genetic technology (using mRNA and DNA), or attenuated virus. Although, these vaccines are able to reduce the severity of the infection and in some cases are able to prevent it, these are not the safest and most compliant technologies for mass vaccination. The vaccines that are based on genetic materials have a high probability of integrating into the human genome. The majority of the candidate vaccines for COVID-19 employ administration of viral antigens or viral gene sequences that aim to induce neutralizing antibodies against the viral spike protein (S), and thereby prevent the uptake through the human ACE2 receptor. However, a growing body of literature highlighting the importance of cellular responses on the recovery of COVID-19 patients has promoted not only the use of vaccine strategies that favor the induction of T cell mediated responses, but also the screening of their production in clinical trial participants. On the other hand, the strategies using whole virus—either attenuated or inactivated—aspire to induce a broader, more heterologous polyclonal antibody response against several viral antigens.

The protein-based vaccines are one of the safest technologies known for vaccine production. The protein-based vaccine strategy eliminates the possibility of severe adverse effect and can be achieved to produce a more durable and stronger immune response by adding a suitable adjuvant. The adjuvant can help in the uptake by adjuvant activated antigen-presenting cells and enhance adaptive immunity. Multiple protein subunit candidates against SARS-CoV-2 are currently in human clinical trials. Each one of these candidates is using different immunogens, principally different forms of the entire spike protein or its receptor binding domain (RBD), the region of the S protein that mediates viral binding to the ACE2 receptor of the target host cells. These proteins still have the risk of binding to the ACE2 receptor potentially creating side effects, as ACE2 receptor is involved in multiple physiological conditions. However, there is a need for a safe and effective protein-based vaccine technology as it is considered as the safer vaccine technology than nucleic acids ones. There is a way to use only immunogenic epitopes for vaccination, minimizing its binding with ACE2. Such epitopes have been specifically identified and embedded in a detoxified version of the tetanus toxin (DrTeNT) protein, specific places to optimize their functions as epitopes. DrTeNT vaccine platform also allows modification in the vaccine to address variants or strains of pathogens for which a vaccine is created. This leads to unique set of recombinant vaccines that need to be expressed and purified.

Thus, there is a need within the vaccine industry for a plasmid for the production of a target protein (e.g. to treat an infection like COVID 19), and/or a mammalian cell or cell population transfected with the plasmid to express the target proteins (e.g. epitopes, antigens, antibodies to elicit an effective immune response against COVID. There is also a need for a method for the production and purification of the target protein, such as through mammalian cell technology.

### SUMMARY OF THE INVENTION

The present invention is directed to a composition, an isolated mammalian cell or cell population, and a vector for transfection into the mammalian cells, and a method for the production of a recombinant protein vaccine in the mammalian cell line. The methods and compositions are particularly useful for generating the stable expression of a recombinant protein vaccine of interest, such as against COVID-19. The invention is particularly useful for the production of vaccines to aid in protection against viral pathogens for vertebrates, in particular mammalians, especially humans.

In an embodiment, the isolated mammalian cell, or cell population, for producing a protein of interest comprises: a plasmid encoded with a nucleotide sequence encoding one or more epitopes or subunits of the SARS-CoV-2 that are embedded in the nucleotide sequence encoding a detoxified recombinant tetanus toxin (DrTeNT).

In one or more embodiments, the nucleotide sequence encoding one or more epitopes or subunits of the SARS-CoV-2 comprises, or consists of, or consists essentially of: SEQ ID NO: 1, or at least 95% sequence identity to SEQ ID NO: 1.

In one or more embodiments, the present invention comprises administering a therapeutic effective amount of the protein/gene encoded within vector, such as to elicit a preventive immune response against an infection or the reduction of one or more signs or symptoms of a disease or medical condition in a subject (e.g., COVID-19, or other known infectious disease).

Accordingly, a first aspect of the presently claimed invention is directed to a process for producing a protein of interest, wherein the process comprises the steps of:
i. culturing a cell (e.g. an isolated mammalian cell or cell population) comprising a plasmid encoded with a nucleotide sequence encoding one or more epitopes or subunits of the SARS-CoV-2 in a medium, such as SEQ ID NO: 1, or at least 95% sequence identity to SEQ ID NO: 1;
ii. expressing the protein of interest; and
iii. harvesting the protein of interest from the mammalian cell or the medium.

The second aspect of the presently claim invention is directed to a process for the purification of the protein of interest comprising at least the steps of:
i. dissolving the protein obtained according to the first aspect at a pH in the range of 5 to 8 to obtain a solution;
ii. dialyzing the solution obtained in step (i) in an appropriate buffer (e.g., Tris-Buffer with 50 mM NaCl containing 1 M urea and protease inhibitor cocktail) of the right pH (e.g., 5-8) to obtain a dialyzed solution;
iii. eluting the dialyzed solution obtained in step (ii.) through a DEAE A50 ion-exchange column using a buffer having a pH in the range of 5 to 8 and a salt concentration in the range of 0 to 500 mM; and
iv. collecting the fraction containing the protein of interest and concentrating to obtain the protein of interest in higher purity.

In an embodiment, step (i.) comprises at least one step of:
a. thawing the cells harvested at a temperature in the range of 0 to 10° C.;
b. washing the thawed cells of step (a.) with TBS buffer;
c. resuspending the washed cells M-per along with the protease inhibitor cocktail at a temperature in the range of 0 to 10° C.;
d. optionally adding tris buffer of 50 mM containing 300 mM of NaCl;
e. adding 1 M urea solution and sonicating while maintaining the temperature in the range of 0 to 10° C.;
f. centrifuging the step (f.) solution to obtain a supernatant;
g. precipitating the supernatant obtained in step (f.) using ammonium sulfate;
h. solubilizing the precipitate obtained in step (g.) in 50 mM Tris-buffer containing 50 mM NaCl, protease inhibitor cocktail and 1 M urea.

The third aspect of the presently claimed invention is directed to an isolated mammalian cell and/or a composition comprising one or more said mammalian cells, for producing a protein of interest, said mammalian cell comprising (e.g., transfected with): a plasmid encoded with a nucleotide sequence encoding one or more epitopes or subunits of the SARS-CoV-2, such as SEQ ID NO: 1, or at least 95% sequence identity to of SEQ ID NO: 1, wherein the nucleotide sequence in expressible format encoding the protein of interest. Hence, the mammalian cells comprising the plasmid are a non-naturally occurring cell.

In a fourth aspect, the nucleotide sequence encoding one or more epitopes or subunits of the SARS-CoV-2, such as SEQ ID NO: 1, or at least 95% sequence identity to SEQ ID NO: 1, is embedded in the backbone of the nucleotide sequence of detoxified recombinant tetanus toxin (DrTeNT).

In a fifth aspect, the process comprises a step of transfecting a plasmid encoded with a nucleotide sequence of one or more epitopes or subunits of the SARS-CoV-2 of SEQ ID NO: 1 (or at least 95% sequence identity to SEQ ID NO: 1) prior to step (i).

In a sixth aspect, the plasmid is cloned with a 6×His-tag at the C-terminal end.

In a seventh aspect, the plasmid is cloned with one or more selection markers, comprising by way of non-limiting examples: Geneticin, Zeocin, Ampicillin, Neomycin, Kanamycin, Puromycin, Hygromycin B, Blasticidin, and/or Mycophenolic acid.

In an eight aspect, the cell or cell population, is an isolated mammalian cell, which is transfected with the plasmids encoded with the nucleotide sequences disclosed herein, said mammalian cell comprising by way of non-limiting examples: mammalian kidney cells, mammalian lung cells, or mammalian ovarian cell or any mammalian suspension or adherent cells, or Chinese hamster ovarian cell.

In a ninth aspect, the step of transfection comprises a step of mixing the plasmid with OptiPro SFM medium and ExpiFectamine prior to mixing with the mammalian cell culturing the cell.

In a tenth aspect, a step comprises adding ExpiFectamine CHO enhancer and Expi-CHO feed to the flask on Day 1 of culturing the mammalian cell.

In an eleventh aspect, a step comprises adding ExpiFectamine CHO enhancer and Expi-CHO feed to the flask on Day 5 of culturing the mammalian cell.

In a twelfth aspect, the protein of interest is harvested on day 5 and/or day 7 and/or day 9 and/or day 11.

In a thirteenth aspect, a step comprises concentrating to obtain the protein of interest in higher purity using ammonium sulfate.

In a fourteenth aspect, the process further comprises a step of dialyzing the solution obtained in step (iv.) in an appropriate buffer of the appropriate pH.

In a fifteenth aspect, an isolated, e.g., non-naturally occurring, mammalian cell comprises: a plasmid encoded with a nucleotide sequence encoding one or more epitopes or subunits of the SARS-CoV-2 of SEQ ID NO: 1, wherein the nucleotide sequence is in an expressible format encoding the protein of interest., and/or additionally, wherein the plasmid is transfected into the mammalian cell.

In a sixteenth aspect, the nucleotide sequence encoding the one or more epitopes or subunits of the SARS-CoV-2 of SEQ ID NO: 1, or at least 95% sequence identity to SEQ ID NO: 1, is embedded in the nucleotide sequence encoding a detoxified recombinant tetanus toxin (DrTeNT).

In a seventeenth aspect, the plasmid is cloned with a 6×His-tag at the C-terminal end.

In an eighteenth aspect, the plasmid is a cloned with at least one selection marker.

In a nineteenth aspect, at least one selection marker is selected from the group consisting of Geneticin, Zeocin, Ampicillin, Neomycin, Kanamycin, Puromycin, Hygromycin B, Blasticidin, and/or Mycophenolic acid.

In a twentieth aspect, the mammalian cell is within a cell culture medium, e.g. a suspension culture.

A twenty-first aspect comprises a pharmaceutical composition comprising one or more mammalian cells, or cell populations disclosed herein (e.g., comprising the plasmids), or one or more proteins expressed by said mammalian cells/population, and a pharmaceutically acceptable carrier.

A twenty-second aspect comprises a method of treatment or prevention against a disorder/disease associated with the protein encoded within the vector, such as an infectious disease.

### BRIEF DESCRIPTION OF THE DRAWINGS

FIG. 1A: illustrates a Western blot of the cell lysate at day 4 for the confirmation of expression of TC2 in Expi CHO cells. (A) TC2 band is clearly visible in between 250 kDa and 150 kDa marker band. Lane 1 and lane 2 are 30 μl and 15 μl load of cell lysate. M is standard marker lane.

FIG. 1B: illustrates the same protein was Western blotted against TC2 polyclonal antibody. Band was visible in between 250 kDa and 150 kDa marker band. Other bands are also visible.

FIG. 2: illustrates the elution profile of TC2 on DEAE A50 column.

FIG. 3A: illustrates an SDS-PAGE gel of eluted protein from DEAE-A 50 column. Lane 1 from left is SDS-PAGE broad range marker (250 kDa to 10 kDa), lane 2 is load, load elute (lane 3-5), 50 mM elute (lane 6), 100 mM elute (lane 7), 150 mM elute (lane 8), 200 mM elute (lane 9), 250 mM (lane 10), 300 mM elute (lane 11-12).

FIG. 3B: illustrates another SDS-PAGE gel of eluted protein from DEAE-A 50 column. Lane 1 is SDS-PAGE broad range marker (250 kDa to 10 kDa), lane 2 is loaded, 350 mM elute (lane 3-5), 400 mM elute (lane 6-8), 450 mM elute (lane 9) and 500 mM elute (lane 10).

FIG. 4: illustrates the results of a Western blot of DEAE A50 eluted fractions.

FIG. 5: illustrates a TC2 (Tetanus Covid 2) Mammalian plasmid.

### DETAILED DESCRIPTION OF THE INVENTION

Before the present compositions, isolated mammalian cells and populations, and methods of the presently claimed invention are described, it is to be understood that this invention is not limited to particular embodiments described, since such embodiments may, of course, vary. It is also to be understood that the terminology used herein is not intended to be limiting, since the scope of the presently claimed invention will be limited only by the appended claims. Furthermore, the particular features, structures or characteristics may be combined in any suitable manner, as would be apparent to a person skilled in the art from this disclosure, in one or more embodiments. Furthermore, while some embodiments described herein include some, but not other features included in other embodiments, combinations of features of different embodiments are meant to be within the scope of the presently claimed invention, and form different embodiments, as would be understood by those in the art. For example, in the appended claims, any of the claimed embodiments can be used in any combination.

Various embodiments of the present invention are based on vaccines that is embedded in the backbone of the nucleotide sequence of detoxified recombinant tetanus toxin (DrTeNT). Methods of making and using a DrTeNT in a vaccine are disclosed, for example, in: PCT/US2021/044054 or WO/2021/248145 A2, entitled “TETANUS VACCINE PLATFORM FOR EMBEDDING COVID-19 VACCINE” and published Dec. 9, 2021; and in U.S. Pat. No. 11,771,752 B2, entitled “Composition for oral or nasal delivery of tetanus, diphtheria, and pertussis vaccine alone or in combination using neurotoxin associated proteins” and issued on Oct. 3, 2023.

Protein Production: Accordingly, an embodiment the presently claim invention is directed to a process for producing a protein of interest, wherein the process comprises the steps of: i) culturing an isolated mammalian cell or cell population comprising a plasmid encoded with a nucleotide sequence encoding one or more epitopes or subunits of the SARS-CoV-2 of SEQ ID NO: 1 (or at least 95% sequence identity to SEQ ID NO: 1) in a medium; ii) expressing the protein of interest; and iii) harvesting the protein of interest from the mammalian cell or the medium.

Protein Purification: the process for the purification of the protein produced comprises at least the steps of:
i) dissolving the protein obtained at a pH in the range of 5 to 8 to obtain a solution;
ii) dialyzing the solution obtained in step (i) in an appropriate buffer of the right pH (e.g., 5-8) to obtain a dialyzed solution;
iii) eluting the dialyzed solution obtained in step (ii.) through a DEAE A50 ion-exchange column using a buffer having a pH in the range of 5 to 8 and a salt concentration in the range of 0 to 500 mM; and
iv) collecting the fraction containing the protein of interest (e.g., the protein encoded by SEQ ID NO: 1) and concentrating to obtain the protein of interest in higher purity.

In an embodiment, step (i.) comprises at least one step of:
a) thawing the cells harvested at a temperature in the range of 0 to 10° C.;
b) washing the thawed cells of step (a.) with TBS buffer;
c) resuspending the washed cells M-per along with the protease inhibitor cocktail at a temperature in the range of 0 to 10° C.;
d) optionally adding tris buffer of 50 mM containing 300 mM of NaCl;
e) Adding 1 M urea solution and sonicating while maintaining the temperature in the range of 0 to 10° C.;
f) centrifuging the step (f.) solution to obtain a supernatant;
g) precipitating the supernatant obtained in step (f.) using ammonium sulfate;
h) solubilizing the precipitate obtained in step (g.) in 50 mM Tris-buffer containing 50 mM NaCl, protease inhibitor cocktail and 1 M urea.

### Nucleotide Sequence for the Expression of the Protein of Interest:

A “vector” is any of a variety of nucleic acids that comprise a desired sequence or sequences to be delivered to and/or expressed in a cell. Vectors are typically composed of DNA, although RNA vectors are also available. In the present invention, a plasmid vector is used in one embodiment, although other vector types are suitable for use herein, e.g., to produce the target protein encoded by SEQ ID NO: 1.

In an embodiment, the present invention comprises an isolated mammalian cell or an isolated mammalian cell population comprising a vector or expression cassette comprising a regulatory region comprising one or more nucleotides (or a polynucleotide) operably linked to a transgene, and wherein said regulatory sequence regulates expression of said transgene.

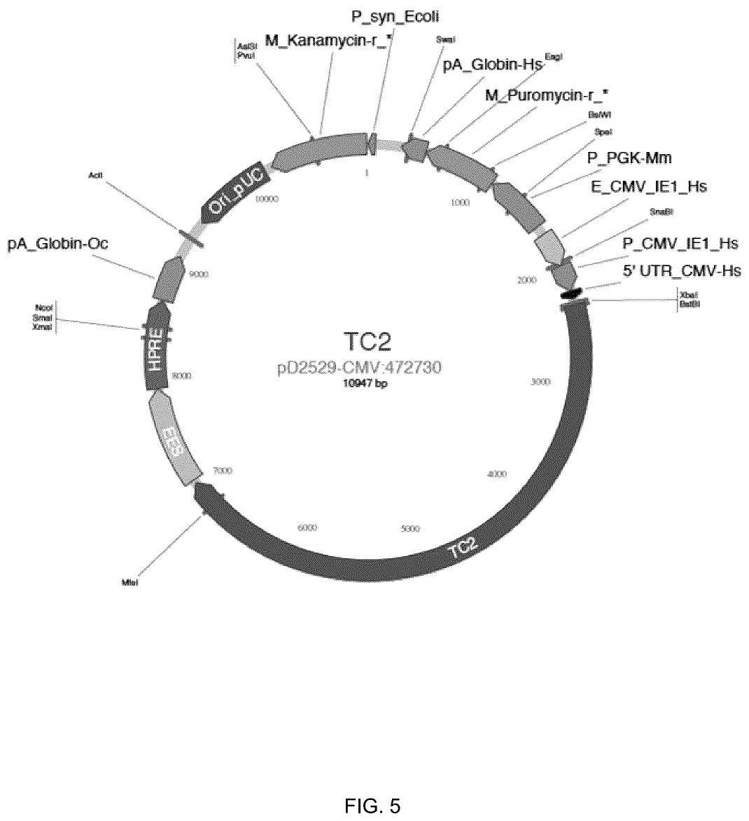

Referring to FIG. 5: In an embodiment, the present invention is directed to a plasmid/vector comprising a nucleotide sequence for the production of a vaccine of interest. In one or more embodiments, the nucleotide sequence encodes one or more epitopes or subunits of the SARS-CoV-2 of SEQ ID NO: 1 (or at least 95% sequence identity to SEQ ID NO: 1) for the production of the vaccine of interest that is embedded in the backbone of the nucleotide sequence of detoxified recombinant tetanus toxin (DrTeNT).

The plasmid comprising the nucleotide sequence for the production of a vaccine of interest is further cloned with a 6×His-tag at the C-terminal end. His-tag is introduced to help in the purification. His-tag is attached to the sequence with thrombin cleavage site, so that it can be removed after purification, if necessary.

The plasmid is further cloned with one or more selection markers. As used herein the term “selection marker” refers to a gene introduced into a cell that confers a trait suitable for artificial selection. Selectable markers for use in the present invention are well-known to those of ordinary skill in the art and include: Geneticin™, Zeocin™, Ampicillin, neomycin, Kanamycin, Puromycin, Hygromycin B, Blasticidin, or Mycophenolic acid. Preferably the one or more selection markers are selected from Kanamycin, or Puromycin. The advantage of using one or more selection markers is to produce stable clones for better yield and easier process for production and purification of the target protein.

### Methods of Treatment:

In an embodiment, the methods of the present invention further comprise administering to a patient a pharmaceutical composition comprising the harvested protein in a pharmaceutical acceptable carrier (a protein-based vaccine), to prevent an infection associated with the harvested protein, or to reduce the adverse effects thereof. In another embodiment, the pharmaceutical composition comprises a population of mammalian cells transfected with a plasmid of the present invention to express the one or more epitopes or subunits of the immunogenic proteins (e.g., SARS-CoV-2 of SEQ ID NO: 1, or at least 95% sequence identity to SEQ ID NO: 1) for in vivo gene therapy.

Routes of administration and dose for both the protein and gene therapy are well known in the art, such as: intramuscular, oral, nasal, etc. The route of administration and dose is selected to deliver a therapeutic effective amount of the protein/gene encoded within vector. A “therapeutically effective” is an amount of the protein/gene/cells sufficient to result in the reduction of one or more signs or symptoms of a disease or medical condition in a subject (e.g., COVID-19, or other known infectious disease).

### Exemplifications

### Culturing the Cell to Produce a Therapeutic Protein:

In an embodiment, the nucleotide sequence (e.g. of the TC2 gene) was meticulously optimized for the mammalian CHO (Chinese Hamster Ovary) cells with kanamycin and puromycin as selection markers. The optimized nucleotide sequence was inserted into the pD2529-CMV-472730 vector by using Xba1, BstB1 and Mfe1 restriction enzymes. The pD2529-CMV-472730 vector was equipped with enhancer/promoter and higher gene expression elements (see FIG. 5). Exemplary expression enhancer sequence (EES) are selected from the group consisting of the full EES, 410-564 EES, and 511-810 EES. And in an embodiment, the promoter is cytomegalovirus (CMV) simian virus 40 early promoter (SV40), human Ubiquitin C promoter (UBC), human elongation factor 1a promoter (EF1A), mouse phosphoglycerate kinase 1 promoter (PGK), chicken β-Actin promoter coupled with CMV early enhancer (CAGG).

The growth condition of CHO cells was optimized as follows. The cells were carefully thawed at 37° C. and drop-wise resuspended into a 125 ml flask containing 25 ml of pre-warmed (at 37° C.) Expi-CHO expression medium (Thermo Fisher Scientific) with 125 rpm at 37° C. and 8% CO2. The cells were grown for 3-4 days and counted using the hemocytometer. The cells were cultured when the total number of cells were in the range of 0.8-1×10⁷ with viability above 95%. For subculturing (passaging), cells were diluted in fresh pre-warmed (at 37° C.) Expi-CHO expression medium having a cell count of ˜5×10⁵ cells. Cells were sub-cultured for three to four passages. The best doubling time for the cells in the growth phase is ˜18 hr±2 hr. If the doubling time was more than 24 hours, then sub-culturing the cells in a fresh media was performed.

The plasmid is purified using a DNA extraction kit well known in the art. The plasmid is embedded with two selection markers, Kanamycin and puromycin. The purposes for embedding selection markers were: a) to get the stronger selection while doing the stable clone selection (if required); and b) to purify the plasmid using bacterial selection marker (kanamycin). The above plasmid was transformed into an E. coli competent cell (BL21(DE3)) and allowed to grow in a 100 ml flask. This was performed using either Qiagen™ DNA extraction kit or Fuji™ DNA extraction kit, and by following the manufacturer's protocol. The yield of the extracted plasmid from 10 ml of culture was ˜500 μg/ml. The plasmid purity was confirmed by running DNA gel.

### Harvesting the Protein of Interest:

The plasmid obtained from the passage that is three or higher was selected for the transfection. Whenever cells are taken out of liquid nitrogen (N₂), then those that need to be transfected were passaged at least two times before the start of transfection. For transfection, cell viability should be more than 95%. The cells were sub-cultured at a cell density of 3×10⁶ cells/ml and grown into a 125 ml flask containing 25 ml of pre-warmed Expi-CHO expression medium (Thermo Fisher Scientific™) with 125 rpm at 37° C. and 8% CO₂ on a day prior to transfection. The next day after counting the cell and checking the viability (>95%), we proceeded with transfection of the plasmid in Expi-CHO cells.

For transfection, 25 μl of plasmid DNA (0.8-1.0 μg/ml) was diluted into 1 ml OptiPro SFM medium (Thermo Fisher Scientific) and kept for 2 min at room temperature, then mixed by inverting the vial gently. 80 μl of Expifectamine reagent was diluted into 920 μl of OptiPro™ SFM media and mixed by gentle inversion (4-5 times). The diluted ExpiFectamine was added to a diluted DNA solution and mixed by gentle swirling or inversion. Incubated for 3 min for complexation and added to the 125 ml flask containing cells in 25 ml media. Incubated the cells in a 37° C. incubator with a humidified atmosphere of 8% CO₂ on an orbital shaker at 120-125 rpm. The cell count was carried out every other day. For getting a better expression of target protein, two protocols at different time points were tried.

Protocol I: Added ExpiFectamine CHO enhancer (6 ml) and Expi-CHO feed (6 ml) to the flask on Day 1 after transfection by gentle swirling. Returned the flask to the incubator at 37° C. and 8% CO₂ with shaking at 120-125 rpm.

### Protocol II:

a) Added ExpiFectamine CHO enhancer (160 μl) and Expi-CHO feed (4 ml) to the flask on Day 1 after transfection by gentle swirling. Returned the flask to the incubator at 37° C. and 5% CO₂ with shaking at 120-125 rpm.
b) On day 5, add ExpiFectamine CHO enhancer (160 μl) and Expi-CHO feed (4 ml) to the flask by gentle swirling. Returned the flask to the incubator at 32° C. and 5% CO₂ with shaking at 120-125 rpm.

The harvesting of the cells was carried out at two time points; day 5 and 9 for protocol I, and day 7 and 9 for protocol II. In both cases, the expression was higher on day 9. The amount of protein was estimated. The day for harvesting was determined based on viability (>70%) and the total number of cells (>3×10⁶). The harvested cells were kept at −80° C. till further processing.

### Purification of the Protein of Interest:

The cells harvested were lysed. The protein was soluble at a higher concentration of the salt, however, when the salt concentration was lowered for ion-exchange column protein starts to precipitate from the solution. The conditions were optimized with the following buffer conditions, including pH and salt concentration. A 50 mM tris buffer at a pH of 7.5 containing 300 mM NaCl with PIC (protease inhibitor cocktail, Roche™) and 1 M urea was used for lysis of the cells. However for the ion exchange column, a salt concentration of 50 mM NaCl was used to dialyze. The protein was completely soluble in this condition.

The protein was purified using two column purification: First Ni-NTA affinity column and then DEAE A50 ion exchange.

In another embodiment the target protein was purified by using only the DEAE A50 ion-exchange column. This single column purification provided the following advantages: a) it is a less tedious process; b) is cost effective (eliminates the need for Ni-NTA affinity column); c) is easily compatible with a scale up processes; and d) requires less time to get the final material.

An exemplary protocol for purification comprised the steps as follows.
(a) The harvested cell pellet was thawed at 4° C., then washed twice with 1×TBS. The pellet was then resuspended in 5 ml M-Per (plus 1 tablet of protease inhibitor cocktail (PIC)) and kept at 4° C. with shaking intermittently for 15 mins. Added 15 ml of 50 mM tris buffer, pH 7.5, containing 300 mM NaCl and one tablet of PIC. Shaked intermittently for 15 minutes.
(b) Added 1 M urea to the above suspension and sonicated for 45 seconds (Power 20 and pulse 50% in sonicator). Sonicated three times on ice. Two minutes interval between two cycles of sonication.
c) After sonication, the suspension was centrifuged for 20 min at 12,000 rpm. Collected the supernatant. The supernatant was further centrifuged for 40 minutes at 12,000 rpm. The centrifugation was performed at a temperature of 4° C.
d) The supernatant was collected and precipitated with ammonium sulfate overnight.
e) The next day, the solution/supernatant was then centrifuged at 12000 rpm for 20 min (at 4° C.). The precipitate was resuspended in a minimum volume of 50 mM Tris-buffer, pH 7.5, containing 50 mM NaCl, PIC and 1M urea. 1M urea helped solubilize the insoluble proteins in lysate.
f) The protein solution was dialyzed using 50 mM Tris buffer, pH 7.5, containing 50 mM NaCl with two changes for two hours. After dialysis PIC and 1 M urea were added according to the final volume and loaded onto pre-equilibrated DEAE A 50 column (˜12-15 cm). Ensured column was equilibrated properly before loading the protein solution. The equilibration buffer was 50 mM Tris, pH 7.5, containing 50 mM NaCl and 1 M urea.
g) The protein was eluted by adding 10 ml of buffer solution with varying NaCl concentrations as shown below in Table 1:

TABLE 1

50 mM tris Buffer pH 7.5 containing 50 mM NaCl
50 mM tris Buffer pH 7.5 containing 100 mM NaCl
50 mM tris Buffer pH 7.5 containing 150 mM NaCl
50 mM tris Buffer pH 7.5 containing 200 mM NaCl
50 mM tris Buffer pH 7.5 containing 250 mM NaCl
50 mM tris Buffer pH 7.5 containing 300 mM NaCl
50 mM tris Buffer pH 7.5 containing 350 mM NaCl
50 mM tris Buffer pH 7.5 containing 400 mM NaCl
50 mM tris Buffer pH 7.5 containing 450 mM NaCl
50 mM tris Buffer pH 7.5 containing 500 mM NaCl

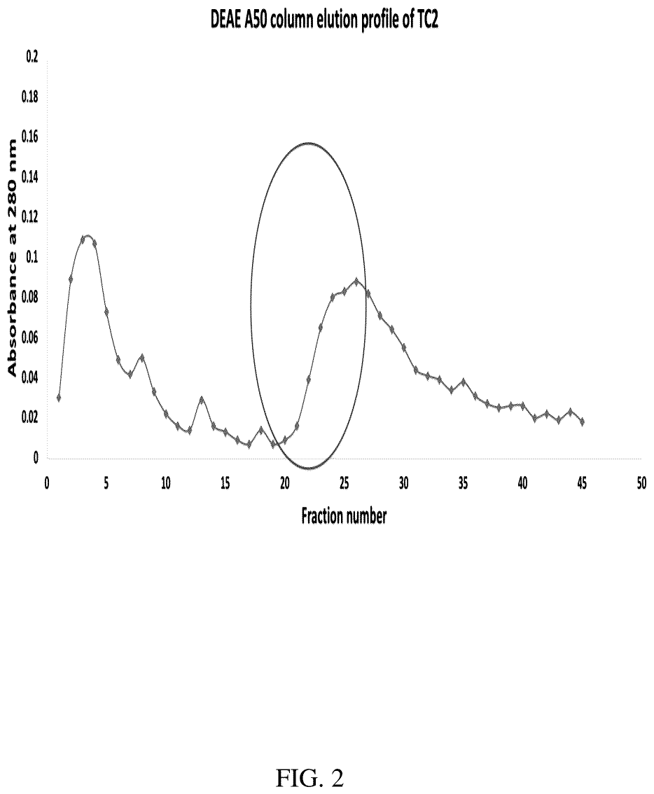

In this protocol the TC2 protein was eluted between 300-400 mM NaCl (fraction no 19-25) (FIGS. 2, 3 and 4). The fractions 19-25 were pooled and concentrated using ammonium sulphate. The final samples were dialyzed against 50 mM tris buffer, pH 7.5, containing 150 mM NaCl and stored at −80° C.

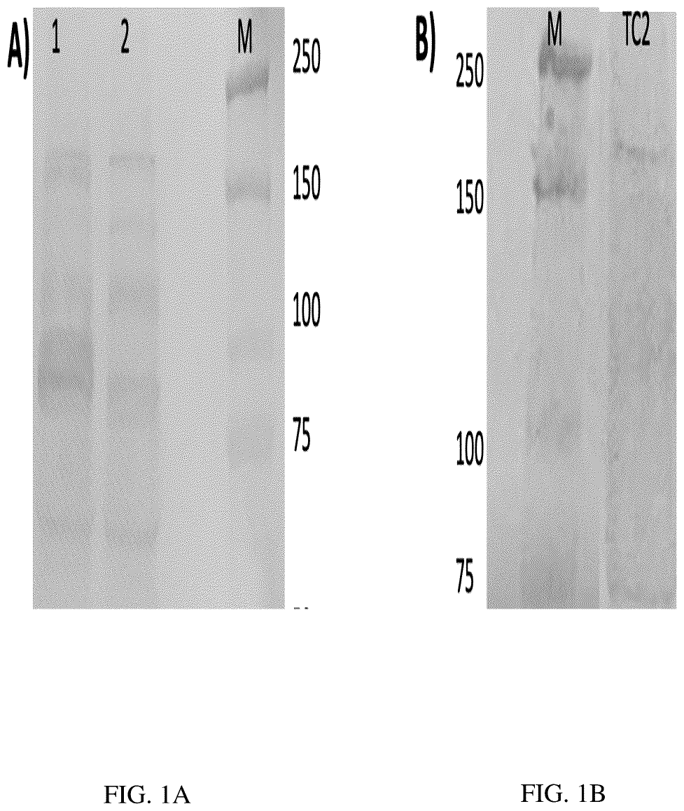

The experimental results are further illustrated in FIGS. 1A-4. FIG. 1A is a Western blot of the cell lysate at day 4 for the confirmation of expression of TC2 in Expi CHO cells. (A) TC2 band is clearly visible in between 250 kDa and 150 kDa marker band. Lane 1 and lane 2 are 30 μl and 15 μl load of cell lysate. M is standard marker lane.

FIG. 1B: illustrates the same protein was visualized through Western blot against TC2 polyclonal antibody. A band was visible in between 250 kDa and 150 kDa marker band. Other bands are also visible that may be the part of TC2 protein, which is recognized by the antibody.

FIG. 2 illustrates the elution profile of TC2 on DEAE A50 column. The fraction number (x-axis) was plotted against the UV absorbance at 280 nm (y-axis).

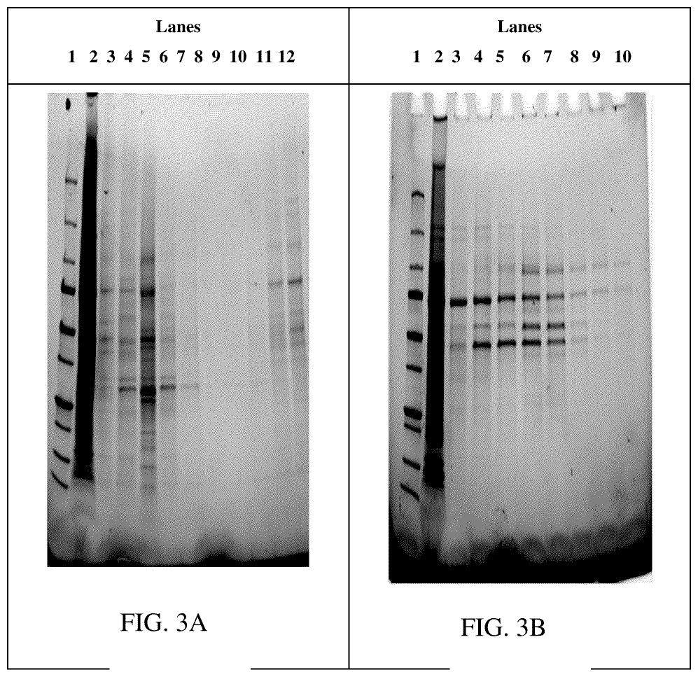

FIG. 3A illustrates an SDS-PAGE gel of eluted protein from an DEAE-A50 column. Lane 1 from left is SDS-PAGE broad range marker (250 kDa to 10 kDa), lane 2 is load, load elute (lane 3-5), 50 mM elute (lane 6), 100 mM elute (lane 7), 150 mM elute (lane 8), 200 mM elute (lane 9), 250 mM (lane 10), 300 mM elute (lane 11-12). 50, 100, 150, 200, 250, 300 mM are the concentration of NaCl in which the respective fractions are eluted.

FIG. 3B also illustrates an SDS-PAGE gel of eluted protein from an DEAE-A50 column Lane 1 is SDS-PAGE broad range marker (250 kDa to 10 kDa), lane 2 is loaded, 350 mM elute (lane 3-5), 400 mM elute (lane 6-8), 450 mM elute (lane 9) and 500 mM elute (lane 10).

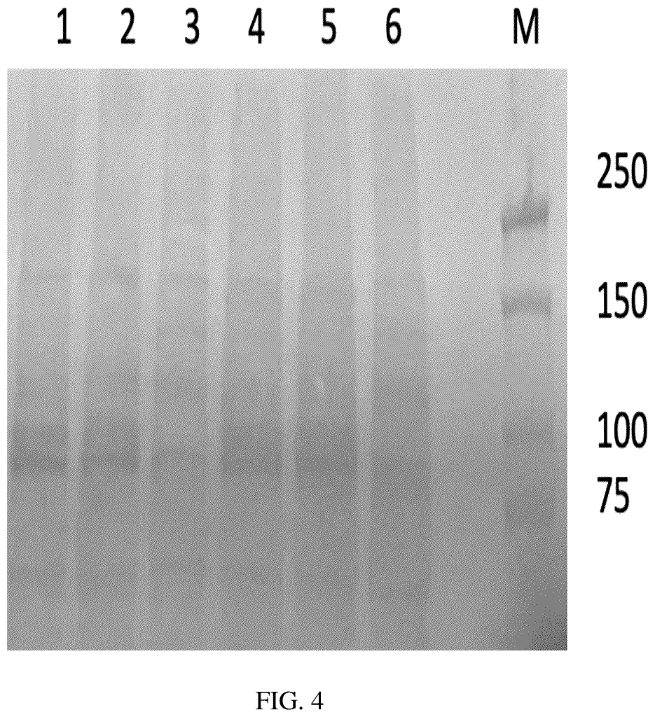

FIG. 4 illustrates the results of a Western blot of DEAE A50 eluted fractions.

### CONCLUSION

The foregoing description of the specific embodiments will so fully reveal the general nature of the embodiments herein that others can, by applying current knowledge, readily modify and/or adapt for various applications such specific embodiments without departing from the generic concept, and, therefore, such adaptations and modifications should and are intended to be comprehended within the meaning and range of equivalents of the disclosed embodiments. It is to be understood that the phraseology or terminology employed herein is for the purpose of description and not of limitation. Therefore, while the embodiments herein have been described in terms of preferred embodiments, those skilled in the art will recognize that the embodiments herein can be practiced with modification within the spirit and scope of the embodiments as described herein.

The transitional term “comprising”, which is synonymous with “including,” “containing,” or “characterized by,” is inclusive or open-ended and does not exclude additional, unrecited elements or method steps. The transitional phrase “consisting of” excludes any element, step, or ingredient not specified in the claim. The transitional phrase “consisting essentially of” limits the scope of a claim to the specified materials or steps “and those that do not materially affect the basic and novel characteristic(s)” of the claimed invention.

Or, the technology illustratively described herein suitably may be practiced in the absence of any element(s) not specifically disclosed herein. Thus, for example, in each instance herein any of the terms “comprising,” “consisting essentially of,” and “consisting of” may be replaced with either of the other two terms.

The term “a” or “an” can refer to one of or a plurality of the elements it modifies (e.g., “a reagent” can mean one or more reagents) unless it is contextually clear either one of the elements or more than one of the elements is described. The term “about” as used herein refers to a value within 5% of the underlying parameter (i.e., plus or minus 1-5%).

As used herein, the term “substantially” refers to approximately the same shape as stated, or synonymous with “about 100%” or the like indicating certainty.

While several embodiments of the disclosure have been described, it is not intended that the disclosure be limited thereto, as it is intended that the disclosure be as broad in scope as the art will allow and that the specification be read likewise. Therefore, the above description should not be construed as limiting, but merely as exemplifications of embodiments.

Trademarks: the product names used in this document are for identification purposes only; and are the property of their respective owners.

The publications, patents and patent applications are incorporated herein by reference. While in the foregoing specification, this invention has been described in relation to certain preferred embodiments thereof, and many details have been set forth for purposes of illustration, it will be apparent to those skilled in the art that the invention is susceptible to additional embodiments and that certain of the details herein may be varied considerably without departing from the basic principles of the invention.

What is claimed is:

1. A process for producing a refined protein of interest, comprising the steps of:
1) culturing an isolated mammalian cell comprising a plasmid encoded with a nucleotide sequence encoding one or more epitopes or subunits of the SARS-CoV-2 of at least 95% sequence identity to SEQ ID NO: 1 in a medium;
2) expressing the protein of interest; and
3) harvesting the protein of interest from the mammalian cell or from the medium.

2. The process according to claim 1, wherein the nucleotide sequence encoding one or more epitopes or subunits of the SARS-CoV-2 of the at least 95% sequence identity to SEQ ID NO: 1 is embedded in the backbone of the nucleotide sequence of detoxified recombinant tetanus toxin (DrTeNT).

3. The process according to claim 1, wherein the plasmid is cloned with a 6×His-tag at a C-terminal end.

4. The process according to claim 1, wherein the plasmid is cloned with one or more identified selection markers.

5. The process according to claim 4, wherein the selection marker comprises one or more: Geneticin, Zeocin, Ampicillin, Neomycin, Kanamycin, Puromycin, Hygromycin B, Blasticidin, or Mycophenolic acid, or any combination thereof.

6. The process according to claim 1, wherein the mammalian cell is suitably selected from mammalian kidney cells, mammalian lung cells, or mammalian ovarian cells.

7. The process according to claim 1, wherein the step of transfection comprises a step of mixing the plasmid in with OptiPro SFM medium and ExpiFectamine prior to mixing with the mammalian cell culturing the cell.

8. The process according to claim 1, further comprising the steps of:
4) dissolving the crude protein obtained after lysis of the mammalian cells obtained according to claim 1, at a pH in the range of 5 to 8 to obtain a solution;
5) dialyzing the solution obtained in step (1) in a buffer of a pH in the range of 5-8 to obtain a dialyzed solution;
6) eluting the dialyzed solution obtained in step (2) through a DEAE A50 ion-exchange column using a buffer having a pH in the range of 5 to 8 and optionally a salt concentration in the range of 0 to 500 mM; and
7) collecting the fraction containing the protein of interest and concentrating said fraction to obtain the protein of interest in higher purity.

9. The process according to claim 8, wherein the step (4) comprises one or more of the following steps:
a. thawing the cells harvested at a temperature in the range of 0 to 10° C.;
b. washing the thawed cells of step (a.) with TBS buffer;
c. resuspending the washed cells M-per along with protease inhibitor cocktail at a temperature in the range of 0 to 10° C.;
d. optionally adding tris buffer, pH 7.5, of 50 mM containing 300 mM of NaCl;
e. adding 1M urea solution and sonicating while maintaining the temperature in the range of 0 to 10° C.;
f. centrifuging the step (e) solution to obtain a supernatant;
g. precipitating the supernatant obtained in step (f.) using optimized amount of ammonium sulphate;
h. solubilizing the precipitate obtained in step (g.) in 50 mM Tris-buffer, pH 7.5, containing 50 mM NaCl, protease inhibitor cocktail and 1M urea.

10. The process according to claim 8, further comprising in step (6) concentrating the solution to obtain the protein of interest in higher purity using a salting out method.

11. The process according to claim 8, wherein the buffer of step (5) for dialyzing the solution comprises Tris-Buffer with 50 mM NaCl containing 1 M urea and protease inhibitor cocktail.

12. An isolated mammalian cell, or an isolated mammalian cell population, for producing a protein of interest, wherein the mammalian cell comprises: a plasmid encoded with a nucleotide sequence encoding one or more epitopes or subunits of the SARS-CoV-2 of SEQ ID NO: 1 or with at least 95% sequence identity to SEQ ID NO. 1, wherein the nucleotide sequence is in expressible format encoding the protein of interest.

13. The mammalian cell according to claim 12, wherein the plasmid encoded with the nucleotide sequence encoding one or more epitopes or subunits of the SARS-CoV-2 of SEQ ID NO: 1 is transfected into the mammalian cell.

14. The mammalian cell according to claim 12, wherein the nucleotide sequence encoding one or more epitopes or subunits of the SARS-CoV-2 of SEQ ID NO: 1 is embedded in the nucleotide sequence encoding a detoxified recombinant tetanus toxin (DrTeNT).

15. The mammalian cell according to claim 12, wherein the plasmid is cloned with a 6×His-tag at a C-terminal end.

16. The mammalian cell according to claim 12, wherein the plasmid is cloned with at least one selection marker.

17. The mammalian cell according to claim 16, wherein the at least one selection markers is selected from the group consisting of Geneticin, Zeocin, Ampicillin, Neomycin, Kanamycin, Puromycin, Hygromycin B, Blasticidin, and Mycophenolic acid.

18. The mammalian cell according to claim 12, wherein the mammalian cell comprises: mammalian kidney cells, mammalian lung cells, or mammalian ovarian cells.

19. The mammalian cell according to claim 12, further comprising the mammalian cell in a cell culture comprising a medium.

20. The mammalian cell according to claim 19, wherein the cell culture is either a suspension culture or an adherent culture.


SEQUENCE LISTING (1 sequence)

SEQ ID NO: 1 - protein, 673 aa, SARS-CoV2
GTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT